(19) United States
(12) Patent Application Publication
(10) Pub. No.: US 2005/0002974 A1
(43) Pub. Date: Jan. 6, 2005

(54) Itch-relieving cosmetic or dermatological preparation

(75) Inventors: Alexander Filbry, Hamburg (DE); Rainer Kropke, Schenefeld (DE); Dagmar Zelle, Bliedersdorf (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG, Hamburg (DE)

(21) Appl. No.: 10/856,004

(22) Filed: May 27, 2004

Related U.S. Application Data
(63) Continuation of application No. PCT/EP02/13033, filed on Nov. 21, 2002.

(30) Foreign Application Priority Data
Nov. 27, 2002 (DE) 101 58 199.8

(51) Int. Cl. A61K 31/555; A61K 6/00

(52) U.S. Cl. 424/401

(57) ABSTRACT

The invention is a cosmetic or dermatological preparation comprising at least one O/W emulsifier, polidocanol, and at least one essential oil. The present invention also includes a cosmetic or dermatological preparation comprising at least one nonionic emulsifier, polidocanol, and menthol. A drug delivery system comprising the dermatological preparation, a dermatological wipe impregnated with the dermatological preparation, and a method for treating the skin or scalp with the dermatological preparation are also included in the present invention.

### CROSS-REFERENCE TO RELATED APPLICATIONS

This is a continuation application of PCT/EP02/13033, filed Nov. 21, 2002, which is incorporated herein by reference in its entirety, and also claims the benefit of German Priority Application No.101 58 199.8, filed Nov. 27, 2001.

### FIELD OF THE INVENTION

The present invention relates to itch-relieving cosmetic and dermatological preparations which include polidocanol and an essential oil. Immediate and long-lasting alleviation of itching is established upon use of the preparation according to the invention.

### BACKGROUND OF THE INVENTION

Itchiness (itching of the skin, pruritus) is a subjectively annoying and unpleasant sensation related to the skin or mucosa. It can occur locally or else spread over the entire body. Itchiness can have a burning, stinging or tingling character and almost always leads to rubbing or scratching. This in turn may lead to skin symptoms such as scratches, open wounds, scabs, and skin infections. The itchiness is perceived via pain receptors in the skin and passed to the brain via the vegetative nerve system. The cause of itchiness is often manifold and can sometimes arise as a first and only symptom in skin disorders or systemic diseases. Besides dry skin, lack of moisture supply, clothing influences, itchiness can, however, also arise as a result of external effects and skin irritation such as, for example, as a result of bites by mosquitoes or following contact with stinging nettles or pain. Itchiness can be a reaction to chemical, mechanical or thermal irritations. It may arise as the result of external irritation, such as, for example, as a result of effects of chemical substances, e.g. histamine (mosquito bite), apamine (bee sting), as a result of allergic immune reaction, as a result of pressure or friction or else as a result of heat or solar irradiation, hives, urticaria and other skin reactions associated with itchiness. Also known and of significance is senile pruritus, which often leads to unpleasant attendant phenomena in elderly people.

For the treatment of itchiness, besides diagnostic cause-finding, itch-reducing or -relieving measures in particular are of prime importance. This often takes place independently of the influence on the underlying disorder.

The known medicaments or alleviating agents for itchiness are administered in tablet form or in the form of powders, ointments, gels or emulsions. Whereas tablets lead to a systemic effect, which affects the entire organism, in the case of gels, creams and emulsions, a specific formulation is necessary in order to keep the active ingredient permanently and stably and primarily in effective form in the preparation.

There are a large number of tried and tested anti-itchiness agents. For example, predominantly cooling gels and sticks are used which, besides cooling as a result of vaporizing alcohol or water, also comprise antihistamines. In addition, isoprenaline is used, although this is not recommended due to known skin irritation.

A known cream is, for example, Camillen 60 FUDES. The cream based on petroleum jelly, calendula oil and menthol for alleviating itchiness. Additives such as antibacterial substances, benzoic acid and triclosan provide preventive protection against fungal attack.

The pain-relieving gel Thesit® sold by Desitin comprises the constituents polidocanol, mepivacain hydrochloride and benzalconium chloride. The gel is intended for the treatment of skin injuries, burns and itchiness, mepivacain hydrochloride acting as a local anesthetic.

DE 19833177 describes the use of a plaster with the active ingredients menthol and benzocain, as local anesthetic, for reducing the irritation of insect bites and contact with stinging nettles. A disadvantage is that local anesthetics are able to trigger contact allergies and the addition of local anesthetic rules out subsequent selling as a cosmetic.

### SUMMARY OF THE INVENTION

An object of the present invention is to provide a preparation which has a long-lasting itch-reducing or -relieving effect, enriches the prior art and offers an alternative to the itch-reducing preparations known to date. A further object is to provide an itch-relieving preparation which does not have the disadvantages of the preparations from the prior art and, in particular, has an immediate long-lasting itch-relieving effect without an excessive amount of itch-relieving active ingredients having to be added. A further object is to provide an itch-relieving preparation which has a care effect and can be applied in a user-friendly manner. It must normalize the moisturization and moisture regulation of the skin and also protect against skin damage as a result of solar irradiation. In particular, in countries with high exposure to the sun, there is in most cases also the risk of insect bites or contact with jellyfish.

It was surprising and could not have been foreseen by the person skilled in the art that a preparation comprising one or more O/W emulsifiers, optionally one or more coemulsifiers, polidocanol and essential oil achieves the set objects.

In particular, the active ingredients polidocanol and menthol in nonionic emulsion bases, in particular in emulsions based on Eumulgin B-1, have proven surprisingly exceptionally effective.

The preparations according to the invention lead to a clearly effective, long-lasting and also subjectively noticeable reduction in the itchiness on the skin.

Polidocanol is an abbreviation for polyethylene glycol (9) monododecyl ether, an adduct of 9 mol of ethylene oxide onto dodecyl alcohol; it corresponds to the formula
C₁₂H₂₅—(O—CH₂—CH₂)ₙ—OH
where n assumes an average value of 9 and the average molar weight is about 600 g/mol. Polidocanol is also known under nonaoxythylene monododecyl ether, PEG-9 lauryl ether, polyethylene glycol 450, lauryl ether polyoxyethylene(9). The trade names and sales companies are, inter alia, Alfonic 1412-9.0 Ethoxylate (Condea Vista), Atlas G-4829 (Uniqema Americas), Calgene Nonionic L-9 (Calgene), Carsonon L-9 (Lonza Inc./Lonza Ltd.), Carsonon L-985 (Lonza Inc./Lonza Ltd.), Hetoxol L-9 (Heterene), Hetoxol LS-9 (Heterene), Jeecol LA-9 (Jeen), Marlipal 24/90 (Condea Chemie (Marl)), Nikkol BL-9EX (Nikko), Procol LA-9 (Protameen), Sympatens-AL/090 (Kolb), and Unicol LA-9 (Universal Preserv-A-Chem).

The emulsifiability of polidocanol is not influenced in the presence of electrolytes. Polidocanol is an amphiphilic compound with high capillary activity. Aqueous solutions exhibit high surface activity and, accordingly, good spreadability on the skin. Used in aqueous solutions, polidocanol exhibits strong local anesthetic properties (K. Söhring et al., Arch. Int. pharmacodyn, 87, 301 [1951]; K. J. Siems and K. Soehring, Arzneim.-Forschung./Drug Res. 2, 109 [1952]; K. Soehring et al., Arch. Int. pharmacodyn. 91, 112 [1952]; H. S. Zipf et al., Arzneim.-Forsch./Drug. Res. 7,162 [1957]).

Polidocanol is used in the preparation in an amount of from 0.01 to 20% by weight, in particular from 1 to 10% by weight.

Among essential oils are known concentrates obtained from plants which are used as natural raw materials primarily in the perfume and foods industries and which consist to a greater or lesser extent of volatile compounds, such as, for example, true essential oils, citrus oils, absolutes, resinoids.

The term is often also used for the volatile ingredients still present in plants. In the actual sense, however, essential oils are understood as being mixtures of volatile components which are prepared by steam distillation from plant raw materials.

True essential oils consist exclusively of volatile components whose boiling point is predominantly between 150 and 300° C. In contrast to, for example, fatty oils, they therefore do not leave behind a residual transparent fatty mark when dabbed onto filter paper. Essential oils comprise primarily hydrocarbons or monofunctional compounds, such as aldehydes, alcohols, esters, ethers and ketones.

Parent compounds are mono- and sesquiterpenes, phenylpropane derivatives and longer-chain aliphatic compounds. In some essential oils, one ingredient predominates (for example eugenol in oil of cloves with more than 85%), others again have an exceptionally complex composition. The organoleptic properties are often not defined by the main components, but by the secondary or trace components, such as, for example, by the 1,3,5-undecatrienes and pyrazines in galbanum oil. In many of the commercially significant essential oils, the number of identified components is in the hundreds. Very many ingredients are chiral, with one enantiomer very often predominating or being exclusively present, such as, for example, (−)-menthol in peppermint oil or (−)-linalyl acetate in lavender oil.

### DETAILED DESCRIPTION OF THE PREFERRED EMBODIMENTS

Menthol has proven to be a particularly preferred essential oil. Menthol is a constituent of the known Japanese peppermint oil (CAS: 20747-49-3). The most important isomer is (−)-menthol. When rubbed into the skin, menthol produces a pleasant cooling feel as a result of surface anesthetization and stimulation of the cold-sensitive nerves in cases of migraine and the like. However, it has been demonstrated that the areas of skin affected have normal or increased temperature.

Further preferred essential oils which may be mentioned are Oleum Eucalypti, Oleum Menthae piperitae, Oleum camphoratum, Oleum Rosmarini, Oleum Thymi, Oleum Pini sibricum and Oleum Pini silverstris and the terpenes 1,8-cineol and levomethanol and Oleum Abietis albae, Oleum Anisi, Oleum Aurantii Floris, Oleum Bergamottae, Oleum Calendulae infusum, Oleum Caryophylli, Oleum Chamomillae, Oleum Cinnamomi ceylanici, Oleum Citri, Oleum Citronellae, Oleum Cupressi, Oleum Cymbopogonis, Oleum Jecoris, Oleum Lavendulae, Oleum Macidis, Oleum Majoranae, Oleum Melaleucae viridiflorae, Oleum Melissae, Oleum Menthae arvensis, Oleum Millefolium, Oleum Myrrhae, Oleum Myrte, Oleum Pini sibricum, Oleum Pinisilvestris, Oleum Salviae, Oleum Santali, Oleum Terebinthinae rectificat., Oleum Valerianae and Oleum Zingiberis.

The essential oils are used individually or in combination with others in an amount of in total 0.001 to 10% by weight, in particular 0.01 to 1% by weight in the preparation.

Frequent application forms of cosmetic or dermatological preparations are finely disperse multiphase systems in which one or more fatty or oil phases are present besides one or more water phases. Of these systems, the actual emulsions are, again the most widespread.

In simple emulsions, finely disperse droplets of one phase surrounded by an emulsifier shell (water droplets in W/O emulsions or lipid vesicles in O/W emulsions) are present in a second phase. The droplet diameters of the customary emulsions are in the range from about 1 μm to about 50 μm. Such “macroemulsions” are, without further coloring additives, milky-white in color and opaque. Finer “macroemulsions”, whose droplet diameters are in the range from about 10⁻¹ μm to about 1 μm, are, again without coloring additives, bluish-white in color and nontransparent.

Only micellar and molecular solutions with particle diameters less than about 10⁻² μm appear clear and transparent.

By contrast, the droplet diameter of transparent or translucent microemulsions is in the range from about 10⁻² μm to about 10⁻¹ μm. Such microemulsions are mostly of low viscosity. The viscosity of many microemulsions of the O/W type is comparable with that of water.

An advantage of microemulsions is that active ingredients may be present in a more finely disperse form in the disperse phase than in the disperse phase of “macroemulsions”. A further advantage is that they are sprayable due to their low viscosity.

A disadvantage of the microemulsions of the prior art is that a high content of one or more emulsifiers must always be used since the small droplet size brings about a large interface between the phases, which usually has to be stabilized by emulsifiers.

The O/W emulsifiers preferred according to the invention include Eumulgin B-1 from Cognis, an emulsion base corresponding to the names polyethylene glycol(12) cetearyl ether, PEG-12 cetyl/stearyl ether, polyethylene glycol 600 cetyl/stearyl ether, polyoxyethylene(12) cetyl/stearyl ether. This compound is also known under the name CETEARETH-12 and is sold under the following names by the following companies: Atlas G4822 (Uniqema Americas), Jeecol CS-12 (Jeen), Procol CS-12 (Protameen), Sabowax CS 11 (Sabo), Sympatens-ACS/120 (Kolb), Unimul-B-1 (Universal Preserv-A-Chem), Volpo CS12 (Croda Oleochemicals).

Likewise advantageous is Myrj 52S from Uniquema, an emulsifier corresponding to the names Macrogol Stearate 2000, Polyethylene Glycol 2000, Monostearate Polyoxyethylene (40), Monostearate Polyoxyl 40, and Stearate Stearethate 40. This compound is also known under the name PEG-40 STEARATE and is sold under the following names by the following companies: AEC PEG-40 Stearate (A & E Connock) Calgene POE (40) MS (Calgene) Crodet S40 (Croda Oleochemicals) Emerest 2715 (Henkel) Emerest 2715 (Henkel/COSPHA) Hetoxamate SA40 (Heterene) Jeemate 2000-DPS (Jeen) Lanoxide-52 (Lanaetex) LIPOPEG-39-S (Lipo) Myrj 52 (Uniqema Americas) Myrj 52S (Uniqema Americas) Nikkol MYS-40 (Nikko) Pegosperse 1750 MS (Lonza Inc./Lonza Ltd.) Protamate 1540 DPS (Protameen) Protamate 2000 DPS (Protameen) Ritox 52 (RITA) ROL 52 (Fabriquimica) Sabowax SE 40 (Sabo) Simulsol M 52 (SEPPIC) Sipoic MS-40 (Specialty Industrial) Sympatens-BS/400 (Kolb) Tego Acid S 40 P (Goldschmidt) Unipeg-S-40 (Universal Preserv-A-Chem).

As coemulsifier, Cutina GMS (Cognis) has proven very advantageous, a coemulsifier corresponding to the name 2,3-Dihydroxypropyl Octadecanoate, Glyceryl 1-stearate, Glyceryl Monostearate, Glycerol 1-Stearate, Jeechem HMS Monostearin, Octadecanoic Acid, 2,3-Dihydroxypropyl Ester Octadecanoic Acid, Monoester with 1,2,3-Propanetriol Stearic Acid 1-Monoglyceride. This compound is also known under the name GLYCERYL STEARATE and is also sold under the names AEC Glyceryl Stearate (A & E Connock) Aldo HMS (Lonza Inc./Lonza Ltd.) Aldo MS (Lonza Inc./Lonza Ltd.) Aldo MSLG (Lonza Inc./Lonza Ltd.) Alkamuls GMS (Rhodia Inc.) Arlacel 129 (Uniqema Americas) Arlacel 161 (Uniqema Americas) Arlacel 169 (Uniquema Americas) Calgene GMS (Calgene) Capmul GMS (Abitec) Ceral Mex (Fabriquimica) Ceral MN (Fabriquimica) Ceral MNT (Fabriquimica) Cerasynt GMS (ISP Van Dyk) Cerasynt SD (ISP Van Dyk) Cithrol GMS N/E (Croda Oleochemicals) CPH-53-N (Hall) CPH-144-N (Hall) Cremophor GS 11 (BASF) Cutina GMS (Cognis) Emerest 2400 (Henkel) Emerest 2400 (Henkel/) ESTOL 1474 (Uniqema (Netherlands)) ESTOL 3740 (Uniqema (Netherlands)) Geleol (Gattefosse s.a.) Imwitor 191 (Condea Chemie (Witten)) Imwitor 900 (Condea Chemie (Witten)) Jeechem GMS-450 (Jeen) Jeechem HMS (Jeen) Kemester 5500 (Witco) Kemester 6000 (Witco) Kessco GMS (Akzo Nobel Surface Chemistry) KESSCO GMS (Stepan) KESSCO GMS 63F (Stepan) KESSCO GMS PURE (Stepan) Lanesta 24 (Lanaetex).

Further O/W emulsifiers or coemulsifiers are polypropoxylated O/W emulsifiers where the polyethoxylated or polypropoxylated O/W coemulsifiers or emulsifiers is or are chosen from the group:
of fatty alcohol ethoxylates of the general formula R—O—(—CH₂—CH₂—O—)ₙ—H where R is a branched or unbranched alkyl, aryl or alkenyl radical and n is a number from 10 to 50,
of ethoxylated wool wax alcohols,
of polyethylene glycol ethers of the general formula R—O—(—CH₂—CH₂—O—)ₙ—R′, where R and R′, independently of one another, are branched or unbranched alkyl or alkenyl radicals and n is a number from 10 to 80,
of fatty acid ethoxylates of the general formula R—COO—(—CH₂—CH₂—O—)ₙ—H, where R is a branched or unbranched alkyl or alkenyl radical and n is a number from 10 to 40,
of etherified fatty acid ethoxylates of the general formula R—COO—(—CH₂—CH₂—O—)ₙ—R′, where R and R′, independently of one another, are branched or unbranched alkyl or alkenyl radicals and n is a number from 10 to 80,
of esterified fatty acid ethoxylates of the general formula R—COO—(—CH₂—CH₂—O—)ₙ—C(O)—R′, where R and R′, independently of one another, are branched or unbranched alkyl or alkenyl radicals and n is a number from 10 to 80,
of polyethylene glycol glycerol fatty acid esters of saturated or unsaturated, branched or unbranched fatty acids and a degree of ethoxylation between 3 and 50,
of ethoxylated sorbitan esters with a degree of ethoxylation of from 3 to 100,
of cholesterol ethoxylates with a degree of ethoxylation between 3 and 50,
of ethoxylated triglycerides with a degree of ethoxylation between 3 and 150,
of alkyl ether carboxylic acids of the general formula R—O—(—CH₂—CH₂—O—)ₙ—CH₂—COOH or cosmetically or pharmaceutically acceptable salts thereof, where R is a branched or unbranched alkyl or alkenyl radical having 5-30 carbon atoms and n is a number from 5 to 30,
of polyoxyethylene sorbitol fatty acid esters, based on branched or unbranched alkanalic or alkenalic acids and having a degree of ethoxylation of from 5 to 100, for example of the sorbeth type,
of alkyl ether sulfates or the acids on which these sulfates are based of the general formula R—O—(—CH₂—CH₂—O—)ₙ—SO₃—H with cosmetically or pharmaceutically acceptable cations, where R is a branched or unbranched alkyl or alkenyl radical having 5-30 carbon atoms and n is a number from 1 to 50,
of fatty alcohol propoxylates of the general formula R—O—(—CH₂—CH(CH₃)—O—)ₙ—H, where R is a branched or unbranched alkyl or alkenyl radical and n is a number from 10 to 80,
of polypropylene glycol ethers of the general formula R—O—(—CH₂—CH(CH₃)—O—)ₙ—R′, where R and R′, independently of one another, are branched or unbranched alkyl or alkenyl radicals and n is a number from 10 to 80,
of propoxylated wool wax alcohols,
of etherified fatty acid propoxylates of the general formula R—COO—(—CH₂—CH(CH₃)—O—)ₙ—R′, where R and R′, independently of one another, are branched or unbranched alkyl or alkenyl radicals and n is a number from 10 to 80,
of esterified fatty acid propoxylates of the general formula R—COO—(—CH₂—CH(CH₃)—O—)ₙ—C(O)—R′, where R and R′, independently of one another, are branched or unbranched alkyl or alkenyl radicals and n is a number from 10 to 80,
of fatty acid propoxylates of the general formula R—COO—(—CH₂—CH(CH₃)—O—)ₙ—H, where R is a branched or unbranched alkyl or alkenyl radical and n is a number from 10 to 80,
of polypropylene glycol glycerol fatty acid esters of saturated or unsaturated, branched or unbranched fatty acids and a degree of propoxylation between 3 and 80,
of propoxylated sorbitan esters with a degree of propoxylation from 3 to 100,
of cholesterol propoxylates with a degree of propoxylation from 3 to 100,
of propoxylated triglycerides with a degree of propoxylation from 3 to 100,
of alkyl ether carboxylic acids of the general formula R—O—(—CH₂—CH(CH₃)—O—)ₙ—CH₂—COOH or cosmetically or pharmaceutically acceptable salts thereof, where R is a branched or unbranched alkyl or alkenyl radical and n is a number from 3 to 50,
of alkyl ether sulfates or the acids on which these sulfates are based of the general formula R—O—(—CH₂—CH(CH₃)—O—)ₙ—SO₃—H with cosmetically or pharmaceutically acceptable cations, where R is a branched or unbranched alkyl or alkenyl radical having 5-30 carbon atoms and n is a number from 1 to 50,
of fatty alcohol ethoxylates/propoxylates of the general formula R—O—Xₙ—Yₘ—H, where R is a branched or unbranched alkyl or alkenyl radical, where X and Y are not identical and in each case are either an oxyethylene group or an oxypropylene group, and n and m, independently of one another, are numbers from 5 to 50,
of polypropylene glycol ethers of the general formula R—O—Xₙ—Yₘ—R′, where R and R′, independently of one another, are branched or unbranched alkyl or alkenyl radicals, where X and Y are not identical and in each case are either an oxyethylene group or an oxypropylene group, and n and m, independently of one another, are numbers from 5 to 100,
of etherified fatty acid propoxylates of the general formula R—COO—Xₙ—Yₘ—R′, where R and R′, independently of one another, are branched or unbranched alkyl or alkenyl radicals, where X and Y are not identical and are in each case either an oxyethylene group or an oxypropylene group, and n and m, independently of one another, are numbers from 5 to 100, and
of fatty acid ethoxylates/propoxylates of the general formula R—COO—Xₙ—Yₘ—H, where R is a branched or unbranched alkyl or alkenyl radical, where X and Y are not identical and in each case are either an oxyethylene group or an oxypropylene group, and n and m, independently of one another, are numbers from 5 to 50.

It is advantageous to choose the fatty alcohol ethoxylates from the group of ethoxylated stearyl alcohols, cetyl alcohols, cetylstearyl alcohols (cetearyl alcohols). Particular preference is given to polyethylene glycol(13) stearyl ether (Steareth-13), polyethylene glycol(14) stearyl ether (Steareth-14), polyethylene glycol(15) stearyl ether (Steareth-15), polyethylene glycol(16) stearyl ether (Steareth-16), polyethylene glycol(17) stearyl ether (Steareth-17), polyethylene glycol(18) stearyl ether (Steareth-18), polyethylene glycol(19) stearyl ether (Steareth-19), polyethylene glycol(20) stearyl ether (Steareth-20), polyethylene glycol(12) isostearyl ether (Isosteareth-12), polyethylene glycol(13) isostearyl ether (Isosteareth-13), polyethylene glycol(14) isostearyl ether (Isosteareth-14), polyethylene glycol(15) isostearyl ether (Isosteareth-15), polyethylene glycol(16) isostearyl ether (Isosteareth-16), polyethylene glycol(17) isostearyl ether (Isosteareth-17), polyethylene glycol(18) isostearyl ether (Isosteareth-18), polyethylene glycol(19) isostearyl ether (Isosteareth-19), polyethylene glycol(20) isostearyl ether (Isosteareth-20), polyethylene glycol(13) cetyl ether (Ceteth-13), polyethylene glycol(14) cetyl ether (Ceteth-14), polyethylene glycol(15) cetyl ether (Ceteth-15), polyethylene glycol(16) cetyl ether (Ceteth-16), polyethylene glycol(17) cetyl ether (Ceteth-17), polyethylene glycol(18) cetyl ether (Ceteth-18), polyethylene glycol(19) cetyl ether (Ceteth-19), polyethylene glycol(20) cetyl ether (Ceteth-20), polyethylene glycol(13) isocetyl ether (Isoceteth-13), polyethylene glycol(14) isocetyl ether (Isoceteth-14), polyethylene glycol(15) isocetyl ether (Isoceteth-15), polyethylene glycol(16) isocetyl ether (Isoceteth-16), polyethylene glycol(17) isocetyl ether (Isoceteth-17), polyethylene glycol(18) isocetyl ether (Isoceteth-18), polyethylene glycol(19) isocetyl ether (Isoceteth-19), polyethylene glycol(20) isocetyl ether (Isoceteth-20), polyethylene glycol(12) oleyl ether (Oleth-12), polyethylene glycol(13) oleyl ether (Oleth-13), polyethylene glycol(14) oleyl ether (Oleth-14), polyethylene glycol(15) oleyl ether (Oleth-15), polyethylene glycol(12) lauryl ether (Laureth-12), polyethylene glycol(12) isolauryl ether (Isolaureth-12), polyethylene glycol(13) cetylstearyl ether (Ceteareth-13), polyethylene glycol(14) cetylstearyl ether (Ceteareth-14), polyethylene glycol(15) cetylstearyl ether (Ceteareth-15), polyethylene glycol(16) cetylstearyl ether (Ceteareth-16), polyethylene glycol(17) cetylstearyl ether (Ceteareth-17), polyethylene glycol(18) cetylstearyl ether (Ceteareth-18), polyethylene glycol(19) cetylstearyl ether (Ceteareth-19), polyethylene glycol(20) cetylstearyl ether (Ceteareth-20).

It is also advantageous to choose the fatty acid ethoxylates from the following group polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20) oleate.

As ethoxylated alkyl ether carboxylic acid or salts thereof, sodium laureth-11 carboxylate can be used advantageously. As alkyl ether sulfate, sodium laureth 1-4 sulfate can be used advantageously. As ethoxylated cholesterol derivative, polyethylene glycol(30) cholesteryl ether can be used advantageously. Polyethylene glycol(25) soyasterol has also proven useful. As ethoxylated triglycerides, the polyethylene glycol(60) evening primrose glycerides can be used advantageously.

It is also advantageous to choose the polyethylene glycol glycerol fatty acid esters from the group consisting of polyethylene glycol(20) glyceryl laurate, polyethylene glycol(21) glyceryl laurate, polyethylene glycol(22) glyceryl laurate, polyethylene glycol(23) glyceryl laurate, polyethylene glycol(6) glyceryl caprate/caprinate, polyethylene glycol(20) glyceryl oleate, polyethylene glycol(20) glyceryl isostearate, and polyethylene glycol(18) glyceryl oleate/cocoate.

It is likewise favorable to choose the sorbitan esters from the group consisting of polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate.

With the preparations according to the invention, an unexpected synergistic active ingredient development arises due to the combination of the active ingredients polidocanol and essential oils, in particular menthol, with the selected O/W emulsifiers.

The selected O/W emulsifiers build a gel network in the coherent aqueous phase in a shape which favors the release of the active ingredients polidocanol and essential oil such that the itch-relieving effect can develop immediately and is nevertheless long-lasting.

The clinical test into the effectiveness of the preparation according to the invention turns out exceptionally positively and confirms the advantageous properties of the preparations. Preparations according to the invention comprising 1% by weight of menthol and 2% by weight of polidocanol, 0.5% by weight of menthol and 2% by weight of polidocanol and a placebo were tested within the framework of a clinically controlled study on a number of subjects aged between 18 and 60 years.

The result:
immediate itch-reduction following application of the preparation
intensification of the itch reduction within 30 minutes following application until virtual complete disappearance of the irritation
after treatment for one week, also visible reduction in the itching
no difference between the preparations according to the invention used

Further additives of the preparations according to the invention are, besides water, customary substances which can be used in cosmetics, as described in more detail below.

As the oil phase of the preparation according to the invention it is possible to use oils with varying polarity, molecular weight and structure. The oil component or the totality of the oil components of the preparation according to the invention is preferably chosen from the group of esters of saturated or unsaturated, branched or unbranched alkane carboxylic acids or hydroxyalkane carboxylic acids with a chain length of from 1 to 44 carbon atoms and saturated or unsaturated, branched or unbranched alcohols with a chain length of from 1 to 44 carbon atoms, from the group of esters of aromatic carboxylic acids and saturated or unsaturated, branched or unbranched alcohols with a chain length of from 1 to 30 carbon atoms if the oil component or the totality of the oil components are a liquid at room temperature. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyidodecyl palmitate, oleyl oleate, oleyl erucate, erucyl oleate, erucyl erucate, and synthetic, semisynthetic and natural mixtures of such esters, e.g. jojoba oil.

The oil phase can advantageously be chosen from the group of branched and unbranched hydrocarbons, of silicone oils, lanolins, of adipic esters, of butylene glycol diesters, of dialkyl ethers or carbonates, the group of saturated or unsaturated, branched alcohols, and fatty acid triglycerides, namely the triglyceryl esters of saturated or unsaturated, branched or unbranched alkane carboxylic acids with a chain length of from 8 to 24, in particular 12-18, carbon atoms. The fatty acid triglycerides can, for example, advantageously be chosen from the group of synthetic, semisynthetic and natural oils, e.g. olive oil, sunflower oil, soybean oil, peanut oil, rapeseed oil, almond oil, palm oil, coconut oil, palm kernel oil and the like.

The oil phase can also advantageously have a content of cyclic or linear silicone oils, or consist entirely of such oils, although it is preferred to use an additional content of other oil phase components apart from the silicone oil or the silicone oils. Cyclomethicone (octamethylcyclotetrasiloxane) is advantageously used as silicone oil to be used according to the invention. However, other silicone oils are also to be used advantageously for the purposes of the present invention, for example hexamethylcyclotrisiloxane, polydimethylsiloxane, poly(methylphenylsiloxane).

For example, dicaprylyl carbonate, dicaprylyl ether, paraffin oil, coco caprylate/caprate, caprylic/capric triglyceride, cyclomethicone, dimethicone, octyldodecanol, and natural oils such as, for example, soybean oil, macadamia oil, evening primrose oil, are very advantageous. In addition, oil mixtures can also be used very advantageously, such as, for example paraffin oil/dicaprylyl ether, paraffin oil/coco caprylate/caprate.

Advantageous oil components are also, for example, butyloctyl salicylate (for example that obtainable under the trade name Hallbrite BHB from CP Hall), hexadecyl benzoate and butyloctyl benzoate and mixtures thereof (Hallstar AB) and diethylhexyl naphthalate (Hallbrite TQ).

The oil components can advantageously be present in a content of from 1 to 50% by weight, based on the total preparation, preference being given to about 1 to 40% by weight.

Through the use of waxes (ester waxes, triglyceride waxes, ethoxylated waxes etc.) in the oil phase or as the oil phase, it is possible to further improve the preparation. It is preferred when the wax component or the totality of the wax components is chosen from the group of esters of saturated or unsaturated, branched or unbranched alkane carboxylic acids or hydroxycarboxylic acids with a chain length of from 1 to 80 carbon atoms and saturated or unsaturated, branched or unbranched alcohols with a chain length of from 1 to 80 carbon atoms, from the group of esters of aromatic carboxylic acids and saturated or unsaturated, branched or unbranched alcohols with a chain length of from 1 to 80 carbon atoms if the wax component or the totality of the wax components are a solid at room temperature, of natural waxes, of diesters of polyols and C10-C80 fatty acids, of ethoxylated waxes, of triglyceride waxes, of C16-C60 fatty acids or C16-C80 fatty alcohols, and of mineral oil waxes.

The wax components can particularly advantageously be chosen from the group of C₁₆₋₃₆-alkyl stearates, of C₁₀₋₄₀-alkyl stearates, of C₂₀₄₀-alkyl isostearates, of C₂₀₋₄₀-dialkyl dimerates, of C₁₈₋₃₈-alkylhydroxystearoyl stearates, of C20-40-dialkyl dimerates, of C₂₀₋₄₀-alkyl erucates, and also C₃₀₋₅₀-alkyl-beeswax, cetyl palmitate, methyl palmitate, cetearyl behenate, octacosanyl stearate. Silicone waxes, such as, for example, stearyltrimethylsilane/stearyl alcohol are also advantageous in some cases.

Also advantageous for the purposes of the present invention are ester waxes which represent esters of
1. saturated or unsaturated, branched or unbranched mono- or dicarboxylic acid having 10 to 50 carbon atoms, preferably 15-45 carbon atoms and
2. glycerol.
   In this connection, mono-, di- and triglycerides may be advantageous.

The glycerides listed below are particularly advantageous:

| Glyceride | Trade name | Available from |
|---|---|---|
| C₁₆₋₁₈-triglyceride | Cremeol HF-52- | Aarhus |
| | SPC | Oliefabrik |
| Glyceryl hydroxystearate | Naturchem GMHS | Rahn |
| Hydrogenated cocoglyceride | Softisan 100 | Huls AG |
| Caprylic/capric/isostearic/adipic | Softisan 649 | Dynamit Nobel |
| triglyceride | | |
| C₁₈₋₃₆ triglyceride | Syncrowax HGLC | Croda GmbH |
| Glyceryl tribehenate | Syncrowax HRC | Croda GmbH |
| Glyceryl tri(12-hydroxystearate) | Thixcin R | Rheox/NRC |
| Hydrogenated castor oil | Cutina HR | Cognis AG |
| C₁₆₋₂₄-triglyceride | Cremeol HF-62- | Aarhus |
| | SPC | Oliefabrik |

It is particularly preferred to choose the wax components from the group of triglyceride waxes, such as C18-38 triglyceride or tribehenin. It has also been found that ethoxylated waxes, such as, for example, PEG-8 beeswax, PEG 6 sorbitan-beeswax, PEG-2 hydrogenated castor oil, and PEG-12 carnauba wax are advantageous.

Furthermore, the preparations according to the invention can comprise dyes or pigments. The dyes and color pigments can be chosen from the corresponding positive list of the Cosmetics Directive or the EC list of cosmetic colorants. In most cases, they are identical to the dyes approved for foods. Pigments may be organic or inorganic in origin, such as, for example, organic ones of the azo type, indigoids, triphenylmethane-like ones, anthraquinones, and xanthine dyes, which are known as D&C and FD&C blues, browns, greens, oranges, reds, yellows. Inorganic pigments consist of insoluble salts of certified dyes, which are referred to as lakes or iron oxides. For example, barium lakes, calcium lakes, aluminum lakes, titanium dioxide, mica and iron oxides can be used. Al salts which may be used are, for example, Red 3 aluminum lake, Red 21 aluminum lake, Red 27 aluminum lake, Red 28 aluminum lake, Red 33 aluminum lake, Yellow 5 aluminum lake, Yellow 6 aluminum lake, Yellow 10 aluminum lake, Orange 5 aluminum lake, Blue 1 aluminum lake and combinations.

Iron oxides or oxide hydrates which are known and in some cases advantageous are, for example, cosmetic yellow oxide C22-8073 (Sunchemical) cosmetic oxide MC 33-120 (Sunchemical), cosmetic brown oxide C33-115 (Nordmann & Rassmann), cosmetic russet oxide C33-8075 (Sunchemical). Ultramarine blue (Les colorants Wacker) can be used as alumosilicate.

Pearlescent pigments can also be incorporated into the emulsions according to the invention. These are known, for example, from the companies Costenoble (Cloisonné grade, Flamenco grade, Low Lustre grade), Merck (Colorona grades, Microna grade, Timiron grade, Colorona, Ronasphere), Les Colorants Wacker (Covapure, Vert oxyde de Chome), Cadre (Colorona, Sicopearl), BASF (Sicopearl, Sicovit), Rona (Colorona). A pearlescent pigment which has proven particularly advantageous is, for example, Timiron Silk Gold and Colorona Red Gold.

Advantageous color pigments are also titanium dioxide, mica, iron oxides (e.g. Fe₂O₃, Fe₃O₄, FeO(OH)) and zinc oxide. Advantageous dyes are, for example, carmine, Prussian blue, chromium oxide green, ultramarine blue and manganese violet. It is particularly advantageous to choose the dyes or color pigments from the following list. (The substances are arranged according to their Color Index Number).

| Color | | |
|---|---|---|
| Index | | CAS No. or |
| Number | Name | empirical formula |
| 10316 | C-ext. Yellow 1 | 846-70-8 |
| 12075 | C-Orange | 3468-63-1 |
| 14700 | C-Red 57 | 4548-53-2 |
| 15510 | C-ext. Orange 8 | 633-96-5 |
| 15585 | C-Red 55 | 2092-56-0 |
| 15585:1 | C-Red 55 | 5160-02-1 |
| 15800:1 | C-ext. Red 57 | 6371-76-2 |
| 15850 | Lithol Rubin 8 | 5858-81-1 |
| 15850:1 | C-Red 12 | 5281-04-9 |
| 15880:1 | C-ext. Red 61 | 6417-83-0 |
| 15980 | C-Orange 9 | C₁₆H₁₀N₂O₇S₂.2Na |
| 15985 | C-Orange 10 | 2783-94-0 |
| 16035 | C-Red 60 | 29956-17-6 |
| 17200 | C-Red 58 | C₁₆H₁₃N₃O₇S₂.2Na |
| 19140 | C-Yellow 10 | 1934-21-0 |
| 20170 | C-ext. Brown 4 | 1320-07-6 |
| | | 6371-84-2 |
| 26100 | C-ext. Red 56 | 85-86-9 |
| 42053 | C-Green 12 | C₃₇H₃₆N₂O₁₀S₃.2Na |
| 42090 | C-Blue 21 | 2650-18-2 |
| 42090 | C-Blue 21 | 2650-18-2 |
| | (ammonium salt) | 6371-85-3 |
| | | 37307-56-5 |
| 45170 | C-Red 59 | 81-88-9 |
| 45170:1 | (Rhodamine B stearate) | C₂₈H₃₁N₂O₃.C₁₈H₃₅O₂ |
| 45370:1 | C-Red 27 | C₂₀H₁₀Br₂O₅ |
| 45380 | C-Red 30 | 17372-87-1 |
| 45380:2 | Tetrabromofluorescein | 15086-94-9 |
| 45410 | C-Red 34 | 18472-87-2 |
| 45410:1 | Tetrabromotetrachlorofluorescein | 13473-26-2 |
| 45425 | C-Red 35 | C₂₀H₁₀I₂O₅.2Na |
| 45425:1 | Fluorescein mixture | 518-40-7 |
| | | 38577-97-8 |
| 47000 | C-ext. Yellow 23 | 8003-22-3 |
| 47005 | C-Yellow 11 | 8004-92-0 |
| 59040 | C-ext. Yellow 24 | 6358-69-6 |
| 60725 | C-ext. Violet 18 | 81-48-1 |
| 61565 | C-Green 10 | 128-80-3 |
| 61570 | C-Green 11 | 4403-90-1 |
| 73360 | C-Red 28 | 2379-74-0 |
| 75120 | C-Orange 12 | 8015-67-6 |
| 75130 | C-Orange 11 | 7235-40-7 |
| 75170 | Guanine | 68-94-0 |
| | | 73-40-50 |
| 75470 | C-Red 50 | C₂₂H₂₀O₁₃ |
| 75480 | Henna | C₁₀H₅O₃. (Lawson) |
| 75810 | C-Green 8 | 11006-34-1 |
| 75810 | C-Green 7 | 479-61-8 |
| | | 519-62-0 |
| 77000 | C-Pigment 1 | Al |
| 77007 | C-Blue 16 | 57455-37-5 |
| 77019 | C-White 11 | 12001-26-2 |
| 77288 | C-Green 9 | 1308-38-9 |
| 77289 | C-Green 14 | 12001-99-9 |
| 77400 | Bronze | 7440-50-8 |
| 77491 | C-Red 45 | 1309-37-1 |
| 77492 | C-Brown 3 | Fe₂O₃FeO(OH) |
| | (C-Yellow 8) | |
| 77499 | C-Black 5 | Fe₃O₄ |
| 77510/20 | C-Blue 17 | C₆FeN₆.4/3 Fe |
| 77742 | C-Violet 11 | 10101-66-3 |
| 77820 | C-Pigment 2 | 7440-22-4 |
| 77891 | C-White 7 | 13463-67-7 (TiO₂) |
| 77947 | C-White 8 | 1314-13-2 |

The dyes and pigments can be present individually or else in a mixture, and be mutually coated with one another, different color effects generally being brought about by different coat thicknesses.

The list of said dyes and color pigments which can be used in the emulsions according to the invention is not of course intended to be limiting.

Preparations according to the invention can also comprise powder substances. Powder substances which are used are, for example, bismuth oxychloride, titanized mica, silicon dioxide (fumed silica), spherical silicon dioxide beads, polymethyl methacrylate beads, micronized Teflon, boron nitride, acrylate polymers, aluminum silicate, aluminum starch octenylsuccinate, bentonite, calcium silicate, cellulose, chalk, corn starch, glyceryl starch, hectorite, hydrated silica, kaolin, magnesium hydroxides, magnesium oxide, magnesium silicates, magnesium trisilicate, maltodextrin, montmorillonite, microcrystalline cellulose, rice starch, silica, talc, mica, titanium dioxide, zinc laurate, zinc myristate, zinc enodecanoate, zinc rosinate, zinc stearate, polyethylene, aluminum oxide, attapulgite, calcium carbonate, calcium silicate, dextran, kaolin, nylon, silica silylate, silk powder, serecite, tin oxide, titanium hydroxide, trimagnesium phosphate, walnut shell powder or any mixtures.

Said customary cosmetic additives allow it to be applied to the skin without a reduction in the effectiveness of the preparation over a prolonged period without externally visible losses.

In addition, further advantageous active ingredients can be incorporated into the preparations. Thus, for example, ribwort tincture has once again proven to increase effectiveness with regard to reducing itching compared with preparations without ribwort tincture.

In addition, care active ingredients can be incorporated, which are not limited to the fat-soluble active ingredients, but can also be chosen from the group of water-soluble active ingredients, for example vitamins and the like. This thus gives rise to a care effect even in cases of prolonged application time.

A surprising property of the preparations according to the invention is that these are very good vehicles for cosmetic or dermatological active ingredients into the skin, preferred active ingredients being antioxidants which can protect the skin against oxidative stress.

The preparations therefore advantageously comprise one or more antioxidants. Favorable, but nevertheless optional, antioxidants which may be used are all antioxidants which are customary or suitable for cosmetic or dermatological applications.

The antioxidants are particularly advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, lipoic acid and derivatives thereof (e.g. dihydrolipoic acid), aurothioglucose, propylthiouracil and other thiols (e.g. thioglycerol, thiosorbitol, thioglycolic acid, thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and also salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol derivatives thereof, vitamin C and derivatives (e.g. ascorbyl palmitate, Mg ascorbyl phosphates, ascorbyl acetates), isoascorbic acid and its derivatives, tocopherols and derivatives (e.g. vitamin E acetate), vitamin A and derivatives (vitamin A palmitate), and coniferyl benzoate of benzoin resin, rutinic acid and derivatives thereof, ferulic acid and derivatives thereof, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of said active ingredients which are suitable according to the invention.

If vitamin E or derivatives thereof are the antioxidant or the antioxidants, it is advantageous to choose their particular concentrations from the range 0.001-10% by weight, based on the total weight of the formulation.

If vitamin A, or vitamin A derivatives, or carotenes or derivatives thereof of the antioxidant or the antioxidants, it is advantageous to choose their particular concentrations from the range 0.001-10% by weight, based on the total weight of the formulation.

According to the invention, active ingredients can also very advantageously be chosen from the group of lipophilic active ingredients, in particular from the following group: acetylsalicylic acid, atropine, azulene, hydrocortisone and derivatives thereof, e.g. hydrocortisone-17 valerate, vitamins, e.g. ascorbic acid and derivatives thereof, vitamins of the B and D series, very favorably vitamin B₁, vitamin B₁₂ and vitamin D₁, but also bisabolol, unsaturated fatty acids, namely the essential fatty acids (often also called vitamin F), in particular γ-linolenic acid, oleic acid, eicosapentanoic acid, docosahexanoic acid and derivatives thereof, chloramphenicol, caffeine, prostaglandins, thyme, camphor, extracts or other products of vegetable and animal origin, e.g. evening primrose oil, borage oil or carob seed oil, fish oils, cod-liver oil, but also ceramides and ceramide-like compounds and so on.

It is also advantageous to choose the active ingredients from the group of refatting substances, for example Purcellinöl®, Eucerit® and Neocerit®.

Particularly advantageously, the active ingredient or the active ingredients are also chosen from the group of NO synthase inhibitors, in particular when the preparations according to the invention are intended for the treatment and prophylaxis of the symptoms of intrinsic or extrinsic skin aging, and also for the treatment and prophylaxis of the harmful effects of ultraviolet radiation on the skin. A preferred NO synthase inhibitor is nitroarginine.

The active ingredient(s) is/are also advantageously chosen from the group which includes catechins and bile esters of catechins and aqueous or organic extracts from plants or parts of plants which have a content of catechins or bile esters of catechins, such as, for example, the leaves of the Theaceae plant family, in particular of the species Camellia sinensis (green tea). Particularly advantageous are typical ingredients thereof (such as e.g. polyphenols or catechins, caffeine, vitamins, sugars, minerals, amino acids, lipids).

Catechins are a group of compounds which are to be regarded as hydrogenated flavones or anthocyanidines and are derivatives of “catechin” (catechol, 3,3′,4′,5,7-flavanpentaol, 2-(3,4-dihydroxyphenyl)chroman-3,5,7-triol). Epicatechin ((2R,3R)-3,3′,4′,5,7-flavanpentaol) is also an advantageous active ingredient for the purposes of the present invention.

Preferred active ingredients are also polyphenols or catechins from the group (−)-catechin, (+)-catechin, (−)-catechin gallate, (−)-gallocatechin gallate, (+)-epicatechin, (−)-epicatechin, (−)-epicatechin gallate, (−)-epigallocatechin and (−)-epigallocatechin gallate.

Flavone and its derivatives (also often collectively called “flavones”) are also advantageous active ingredients for the purposes of the present invention. They are characterized by the following basic structure (substitution positions are shown):

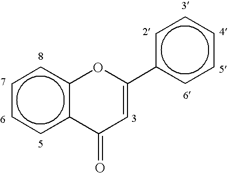

Some of the more important flavones which can also preferably be used in preparations according to the invention are given in the table below:

| | OH substitution positions | | | | | | | |
|---|---|---|---|---|---|---|---|---|
| | 3 | 5 | 7 | 8 | 2′ | 3′ | 4′ | 5′ |
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kaempferol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones are usually in glycosylated form.

According to the invention, the flavonoids are preferably chosen from the group of substances of the generic structural formula 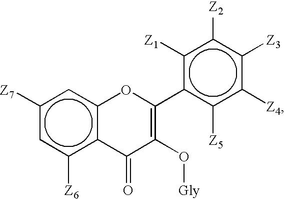
where Z₁ to Z₇, independently of one another, are chosen from the group consisting of H, OH, alkoxy and hydroxyalkoxy, where the alkoxy and hydroxyalkoxy groups can be branched or unbranched and have 1 to 18 carbon atoms, and where Gly is chosen from the group of mono- and oligoglycoside radicals.

According to the invention, the flavonoids can however, also advantageously be chosen from the group of substances of the generic structural formula 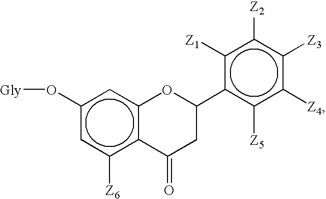
where Z₁ to Z₆, independently of one another, are chosen from the group consisting of H, OH, alkoxy and hydroxyalkoxy, where the alkoxy and hydroxyalkoxy groups can be branched or unbranched and have 1 to 18 carbon atoms, and where Gly is chosen from the group of mono- and oligoglycoside radicals.

Preferably, such structures can be chosen from the group of substances of the generic structural formula 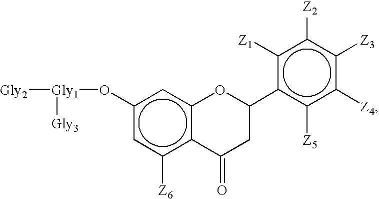
where Gly₁, Gly₂ and Gly₃, independently of one another, are monoglycoside radicals or Gly₂ and Gly₃ can also, individually or together, represent saturations by hydrogen atoms.

Preferably, Gly₁, Gly₂ and Gly₃, independently of one another, are chosen from the group of hexosyl radicals, in particular of rhamnosyl radicals and glucosyl radicals. However, other hexosyl radicals, for example allosyl, altrosyl, galactosyl, gulosyl, idosyl, mannosyl and talosyl, can also be used advantageously in some circumstances. It may also be advantageous according to the invention to use pentosyl radicals.

Z₁ to Z₅ are, independently of one another, advantageously chosen from the group consisting of H, OH, methoxy, ethoxy and 2-hydroxyethoxy, and the flavone glycosides have the structure

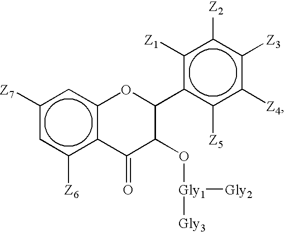

The flavone glycosides according to the invention are particularly advantageously from the group given by the following structure: 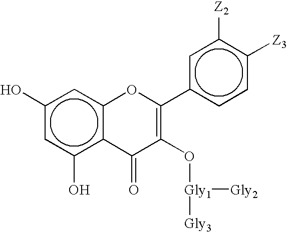
where Gly₁, Gly₂ and Gly₃, independently of one another, are monoglycoside radicals or. Gly₂ and Gly₃ can also, individually or together, represent saturations by hydrogen atoms.

Preferably, Gly₁, Gly₂ and Gly₃, independently of one another, are chosen from the group of hexosyl radicals, in particular of rhamnosyl radicals and glucosyl radicals. However, other hexosyl radicals, for example allosyl, altrosyl, galactosyl, gulosyl, idosyl, mannosyl and talosyl, can also advantageously be used in some circumstances. It may also be advantageous according to the invention to use pentosyl radicals.

For the purposes of the present invention, it is particularly advantageous to choose the flavone glycoside(s) from the group consisting of α-glucosylrutin, α-glucosylmyricetin, α-glucosylisoquercitrin, α-glucosylisoquercetin and α-glucosylquercitrin. Particular preference is given, according to the invention, to α-glucosylrutin.

Also advantageous according to the invention are naringin (aurantin, naringenin-7-rhamnoglucoside), hesperidin (3′,5,7-trihydroxy-4′-methoxyflavanone-7-rutinoside, hesperidoside, hesperetin-7-O-rutinoside), rutin (3,3′,4′,5,7-pentahydroxyflyvone-3-rutinoside, quercetin-3-rutinoside, sophorin, birutan, rutabion, taurutin, phytomelin, melin), troxerutin (3,5-dihydroxy-3′,4′,7-tris(2-hydroxyethoxy)flavone-3-(6-O-(6-deoxy-α-L-mannopyranosyl )-β-D-glucopyranoside)), monoxerutin (3,3′,4′,5-tetrahydroxy-7-(2-hydroxyethoxy)flavone-3-(6-O-(6-deoxy-α-L-mannopyranosyl)-β-D-glucopyranoside)), dihydrorobinetin (3,3′,4′,5′,7-pentahydroxyflavanone), taxifolin (3,3′,4′,5,7-pentahydroxy-flavanone), eriodictyol-7-glucoside (3′,4′,5,7-tetrahydroxyflavanone-7glucoside), flavanomarein (3′,4′,7,8-tetrahydroxyflavanone-7glucoside) and isoquercetin (3,3′,4′,5,7-pentahydroxyflavanone-3-(β-D-glucopyranoside).

It is also advantageous to choose the active ingredient(s) from the group of ubiquinones and plastoquinones.

Ubiquinones are distinguished by the structural formula 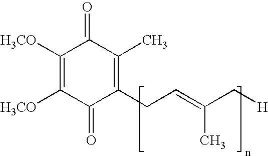
and are the most widespread and thus the most investigated bioquinones. Ubiquinones are referred to depending on the number of isoprene units linked in the side chain as Q-1, Q-2, Q-3 etc., or depending on the number of carbon atoms, as U-5, U-10, U-15 etc. They preferably appear with certain chain lengths, e.g. in some microorganisms and yeasts where n=6. In most mammals including man, Q10 predominates.

Coenzyme Q10 is particularly advantageous and is characterized by the following structural formula:

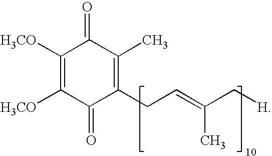

Plastoquinones have the general structural formula

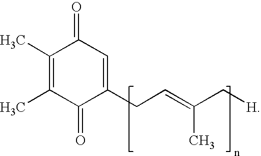

Plastoquinones differ in the number n of isoprene radicals and are referred to accordingly, e.g. PQ-9 (n=9). In addition, other plastoquinones with varying substituents on the quinone ring exist.

Creatine or creatine derivatives are preferred active ingredients for the purposes of the present invention. Creatine is characterized by the following structure:

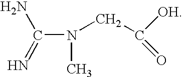

Preferred derivatives are creatine phosphate and creatine sulfate, creatine acetate, creatine ascorbate and the derivatives esterified at the carboxyl group with mono- or polyfunctional alcohols.

A further advantageous active ingredient is L-carnitine [3-hydroxy-4-(trimethylammonio)butyrobetaine]. Acylcarnitines which chosen from the group of substances of the following general structural formula 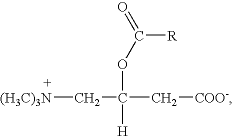
where R is chosen from the group of branched and unbranched alkyl radicals having up to 10 carbon atoms, are also advantageous active ingredients for the purposes of the present invention. Preference is given to propionylcarnitine and, in particular, acetylcarnitine. Both enantiomers (D and L form) are to be used advantageously for the purposes of the present invention. It may also be advantageous to use any enantiomer mixtures, for example a racemate of D and L form. Further advantageous active ingredients are sericoside, pyridoxol, aminoguadine, phytochelatin, isoflavones (genistein, daidzein, daidzin, glycitin), niacin, tyrosine sulfate, dioic acid, adenosine, pyridoxine, arginine, vitamin K, biotin and aroma substances, sericoside, such as also active ingredient combinations of said active ingredients.

Moreover, one of the most important tasks of cosmetic or dermatological preparations is the moisturization and moisture regulation of the skin. For this purpose, besides water, so-called moisturizers are added to the preparations as a constituent of all emulsions. Synthetic moisturizers are replacements for the natural moisturizing factor (NMF), which consists of 40% of free amino acids, 12% of pyroglutamic acid, 12% of lactates, 7% of urea, 1.5% of uric acid, and also glucosamine, creatinine and various salts. Besides hydrolyzed proteins, the synthetic moisturizers used are primarily polyols (polyhydric alcohols).

The most important representative of the polyols is glycerol (1,2,3-propanetriol), a colorless and odorless, sweet-tasting liquid. Glycerol has the following structure:

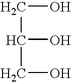

A further important representative of the polyols is sorbitol, a pentavalent alcohol which occurs in rowanberries and can be obtained synthetically by reducing glucose. Sorbitol is characterized by the following structure:

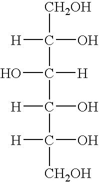

In particular, moisturizers chitosan, fucogel, lactic acid, propylene glycol, sorbitol, polyethylene glycol, dipropylene glycol, butylene glycol, mannitol, sodium pyrrolidone carboxylic acid, glycine hyaluronic acid and salts thereof, amino acids, such as glycine, urea, sodium and potassium salts can also be joined to the skin more easily. Through the use of waxes (ester waxes, triglyceride waxes, ethoxylated waxes etc.) in the oil phase or as the oil phase, adhesion of the active ingredients and moisturizers can be further improved.

The moisture content of the skin can be determined by means of corneometric measurements. A corneometer is used here to investigate the dielectric properties of the stratum corneum. The corneometer consists of a scatter capacitor whose capacity is (co)determined by the dielectric properties of the stratum corneum. In order to ascertain how long the skin moisturization brought about by a cosmetic or dermatological preparation lasts, the moisture content of the skin is determined under constant measurement conditions in each case prior to use and also two hours after use of the cosmetic or dermatological preparation. It has been found in these investigations that the preparation according to the invention comprising moisturizer has a positive influence on the moisture balance of the skin, which is extremely pleasant for skin damaged by skin irritation.

The list of specified additional active ingredients or active ingredient combinations which can be used in the preparations according to the invention is not of course intended to be limiting. The active ingredients can be used individually or in any combinations with one another.

It is in some cases possible and advantageous to use the preparations according to the invention as bases for pharmaceutical formulations. Corresponding requirements apply mutatis mutandis to the formulation of medicinal preparations. The transitions between pure cosmetics and pure pharmaceuticals are fluid here. Suitable pharmaceutical active ingredients according to the invention are in principle all classes of active ingredient, preference being given to lipophilic active ingredients. Examples are: antihistamines, antiphlogistics, antibiotics, antimycotics, the active ingredients which promote circulation, keratolytics, antihistamines, antiphlogistics, antibiotics, antimycotics, the active ingredients which promote circulation, keratolytics, hormones, steroids, vitamins, hormones, steroids, vitamins etc.

It is in some instances possible and advantageous to incorporate repellents into the preparations according to the invention. Particularly advantageous repellent active ingredients for the purposes of the present invention are the abovementioned active ingredients N,N-diethyl-3-methylbenzamide, ethyl 3-(N-n-butyl-N-acetylamino)propionate, 2-(2-hydroxyethyl)-1-methylpropyl 1-piperidinecarboxylate and dimethyl phthalate.

It is also advantageous for the purposes of the present invention to create cosmetic and dermatological preparations whose purpose is also protection against sunlight. For example UV-A and/or UV-B filter substances are usually incorporated. UV protectants, like antioxidants and, if desired, preservatives, also represent effective protection of the preparations themselves against spoilage. Accordingly, the preparations for the purposes of the present invention preferably comprise at least one UV-A, UV-B or broadband filter substance. The formulations may, but do not necessarily, optionally also comprise one or more organic or inorganic pigments as UV filter substances, which may be present in the water phase or the oil phase.

For the purposes of the present invention, particularly advantageous UV filter substances which are liquid at room temperature are homomenthyl salicylate (INCI: Homosalicylate), 2-ethylhexyl 2-cyano-3,3-diphenylacrylate (INCI: Octocrylene), 2-ethylhexyl 2-hydroxybenzoate (2-ethylhexyl salicylate, ethylhexyl salicylate, INCI: Ethylhexyl Salicylate) and esters of cinnamic acid, preferably (2-ethylhexyl) 4-methoxycinnamate (INCI: Ethylhexyl Methoxycinnamate) and isopentyl 4-methoxycinnamate (INCI: Isoamyl p-Methoxycinnamate), 3-(4-(2,2-bisethoxycarbonylvinyl)phenoxy)propenyl)methoxysiloxane/dimethylsiloxane copolymer, which is available, for example, under the trade name Parsol® SLX from Hoffmann La Roche.

Preferred inorganic pigments are metal oxides or other metal compounds which are insoluble or sparingly soluble in water, in particular oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides, and the sulfate of barium (BaSO₄).

For the purposes of the present invention, the pigments can advantageously also be used in the form of commercially available oily or aqueous predispersions. Dispersion auxiliaries or solubilization promoters may advantageously be added to these predispersions.

According to the invention, the pigments may advantageously be surface-treated (“coated”), the intention being to form or retain, for example, a hydrophilic, amphiphilic or hydrophobic character. This surface treatment can consist in providing the pigments with a thin hydrophilic or hydrophobic inorganic or organic coat by methods known per se. The various surface coatings may also comprise water for the purposes of the present invention.

Inorganic surface coatings for the purposes of the present invention may consist of aluminum oxide (Al₂O₃), aluminum hydroxide Al(OH)₃, or aluminum oxide hydrate (also: alumina, CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9), or iron oxide (Fe₂O₃). These inorganic surface coatings may be present on their own, in combination, or in combination with organic coating materials.

For the purposes of the present invention, organic surface coatings may consist of vegetable or animal aluminum stearate, vegetable or animal stearic acid, lauric acid, dimethylpolysiloxane (also: dimethicone), methylpolysiloxane (methicone), simethicone (a mixture of dimethylpolysiloxane with an average chain length of from 200 to 350 dimethylsiloxane units and silica gel) or alginic acid. These organic surface coatings may be present on their own, in combination, or in combination with inorganic coating materials.

Zinc oxide particles and predispersions of zinc oxide particles which are suitable according to the invention are available under the following trade names from the companies listed:

| Trade name | Coating | Manufacturer |
|---|---|---|
| Z-Cote HP1 | 2% dimethicone | BASF |
| ZnO NDM | 5% dimethicone | H&R |
| MZ-303S | 3% methicone | Tayca Corporation |
| MZ-505S | 5% methicone | Tayca Corporation |

Suitable titanium dioxide particles and predispersions of titanium dioxide particles are available under the following trade names from the companies listed:

| Trade-name | Coating | Manufacturer |
|---|---|---|
| MT-100 V | aluminum hydroxide/stearic acid | Tayca |
| | | Corporation |
| MT-100Z | aluminum hydroxide/stearic acid | Tayca |
| | | Corporation |
| Eusolex T-2000 | alumina/simethicone | Merck KgaA |
| Titanium dioxide T805 | octyltrimethylsilane | Degussa |
| (Uvinul TiO₂) | | |
| MT-100AQ | silica/aluminum hydroxide/ | Tayca |
| | alginic acid | Corporation |
| Eusolex T-Aqua | water/alumina/sodium | Merck KgaA |
| | metaphosphate | |

Advantageous UV-A filter substances for the purposes of the present invention are dibenzoylmethane derivatives, in particular 4-(tert-butyl)-4′-methoxydibenzoylmethane (CAS No. 70356-09-1), which is sold by Givaudan under the name Parsol® 1789 and by Merck under the trade name Eusolex® 9020.

For the purposes of the present invention, advantageous further UV filter substances are sulfonated, water-soluble UV filters, such as, for example:
phenylene-1,4-bis(2-benzimidazyl)-3,3′-5,5′-tetrasulfonic acid and its salts, particularly the corresponding sodium, potassium or triethanolammonium salts, in particular the phenylene-1,4-bis(2-benzimidazyl)-3,3′-5,5′-tetrasulfonic acid bis-sodium salt with the INCI name Bisimidazylate (CAS No.: 180898-37-7), which is available, for example, under the trade name Neo Heliopan A P from Haarmann & Reimer;
salts of 2-phenylbenzimidazol-5-sulfonic acid, such as its sodium, potassium or its triethanolammonium salt, and the sulfonic acid itself with the INCI name phenylbenzimidazole sulfonic acid (CAS No. 27503-81-7), which is available, for example, under the trade name Eusolex 232 from Merck or under Neo Heliopan Hydro from Haarmann & Reimer;
1,4-di(2-oxo-10-sulfo-3-bornylidenemethyl)benzene (also: 3,3′-(1,4-phenylene-dimethylene)bis(7,7-dimethyl-2-oxobicyclo[2.2.1]hept-1-ylmethanesulfonic acid) and salts thereof (particularly the corresponding 10-sulfato compounds, in particular the corresponding sodium, potassium or triethanolammonium salt), which is also referred to as benzene-1,4-di(2-oxo-3-bornylidenemethyl-10-sulfonic acid). Benzene-1,4-di(2-oxo-3-bornylidenemethyl-10-sulfonic acid) has the INCI name Terephthalidene Dicamphor Sulfonic Acid (CAS No.: 90457-82-2) and is available, for example, under the trade name Mexoryl SX from Chimex;
sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)-sulfonic acid and salts thereof.

Advantageous UV filter substances for the purposes of the present invention are also so-called broadband filters, i.e. filter substances which absorb both UV-A and also UV-B radiation.

Advantageous broadband filters or UV-B filter substances are, for example, triazine derivatives, such as, for example,
2,4-bis{[4-(2-ethylhexyloxy)-2-hydroxy]phenyl}-6-(4-methoxyphenyl)-1,3,5-triazine (INCI: Bisethylhexyloxyphenol Methoxyphenyl Triazine), which is available under the trade name Tinosorb® S from CIBA-Chemikalien GmbH;
diethylhexylbutylamidotriazone (INCI: Diethylhexyl Butamido Triazone), which is available under the trade name UVASORB HEB from Sigma 3V;
tris(2-ethylhexyl)4,4′,4″-(1,3,5-triazine-2,4,6-triyltriimino)trisbenzoate, also: 2,4,6-tris[anilino(p-carbo-2′-ethyl-1′-hexyloxy)]-1,3,5-triazine (INCI: Ethylhexyl Triazone), which is sold by BASF Aktiengesellschaft under the trade name UVINUL® T 150.

Further triazine derivatives advantageous for the purposes of the present invention are also 2,4-bis{[4-(3-sulfonato)-2-hydroxypropyloxy)-2-hydroxy]phenyl}-6-(4-methoxyphenyl)-1,3,5-triazine sodium salt, 2,4-bis{[4-(3-(2-propyloxy)-2-hydroxypropyloxy)-2-hydroxy]phenyl}-6-(4-methoxyphenyl)-1,3,5-triazine, 2,4-bis{[4-(2-ethylhexyloxy)-2-hydroxy]phenyl}-6-[4-(2-methoxyethylcarboxyl)phenylamino]-1,3,5-triazine, 2,4-bis{[4-(3-(2-propyloxy)-2-hydroxypropyloxy)-2-hydroxy]phenyl}-6-[4-(2-ethylcarboxyl)phenylamino]-1,3,5-triazine, 2,4-bis{[4-(2-ethylhexyloxy)-2-hydroxy]-phenyl}-6-(1-methylpyrrol-2-yl)-1,3,5-triazine, 2,4-bis{[4-tris(trimethylsiloxysilylpropyl-oxy)-2-hydroxy]phenyl}-6-(4-methoxyphenyl)-1,3,5-triazine, 2,4-bis{[4-(2″-methyl-propenyloxy)-2-hydroxy]phenyl}-6-(4-methoxyphenyl)-1,3,5-triazine and 2,4-bis{[4-(1′,1′,1′,3′,5′,5′,5′-heptamethylsiloxy-2″-methylpropyloxy)-2-hydroxy]phenyl}-6-(4-methoxyphenyl)-1,3,5-triazine.

An advantageous benzotriazole for the purposes of the present invention is 2,2′-methylenebis(6-(2H-benzotriazol-2-yl)4-(1,1,3,3-tetramethylbutyl)phenol), a broadband filter which is characterized by the chemical structural formula 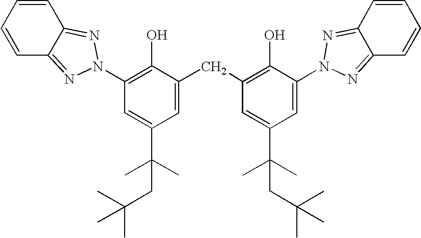
and is available under the trade name Tinosorb® M from CIBA-Chemikalien GmbH.

An advantageous benzotriazole for the purposes of the present invention is, also 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) with the INCI name Drometrizole Trisiloxane, which is characterized by the chemical structural formula

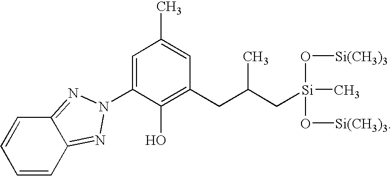

Further advantageous benzotriazoles for the purposes of the present invention are [2,4′-dihydroxy-3-(2H-benzotriazol-2-yl)-5-(1,1,3,3-tetramethylbutyl)-2′-n-octoxy-5′-benzoyl]diphenylmethane, 2,2′-methylenebis[6-(2H-benzotriazol-2-yl)-4-(methyl)phenol], 2,2′-methylenebis[6-(2H-benzotriazol-2-yl)-4-(1,1,3,3-tetramethylbutyl)phenol], 2-(2′-hydroxy-5′-octylphenyl)benzotriazole, 2-(2′-hydroxy-3′,5′-di-t-amylphenyl)benzotriazole and 2-(2′-hydroxy-5′-methylphenyl)benzotriazole.

The further UV filter substances may be oil-soluble or water-soluble.

Advantageous oil-soluble UV-B or broadband filter substances for the purposes of the present invention are, for example:
3-benzylidenecamphor derivatives, preferably 3-(4-methylbenzylidene)camphor, 3-benzylidenecamphor;
4-aminobenzoic acid derivatives, preferably 2-ethylhexyl 4-(dimethylamino)benzoate, amyl 4-(dimethylamino)benzoate;
3-(4-(2,2-bisethoxycarbonylvinyl)phenoxy)propenyl)methoxysiloxane/dimethylsiloxane copolymer, which is available, for example, under the trade name Parsol® SLX from Hoffmann La Roche.

A further photoprotective filter substance to be used advantageously according to the invention is ethylhexyl 2-cyano-3,3-diphenylacrylate (Octocrylene), which is available from BASF under the name Uvinul® N 539.

It has also been found that hydroxybenzophenones, in particular aminobenzophenones, in combination with acrylamide polymers or copolymers, are suitable photoprotective filters and may therefore be advantageously present in the preparation according to the invention.

The list of said UV filters which may be used for the purposes of the present invention is not of course intended to be limiting.

Advantageously, the preparations according to the invention comprise the substances which absorb UV radiation in the UV-A and/or UV-B region in a total amount of, for example, 0.1% by weight to 30% by weight, preferably 0.5 to 20% by weight, in particular 1.0 to 15.0% by weight, in each case based on the total weight of the preparations, in order to provide cosmetic preparations which protect the skin from the entire range of ultraviolet radiation.

The cosmetic and dermatological preparations according to the invention can comprise cosmetic auxiliaries, as are customarily used in such preparations, for example preservatives, preservative aids, bactericides, perfumes, antifoam substances, thickeners and fillers, which improve the feel on the skin.

The application form of the preparation according to the invention can be adjusted as desired as a gel, cream, lotion, spray, as drug delivery system or by means of impregnated wipes.

In the technical sense, gels means: relatively dimensionally stable, readily deformable disperse systems of at least two components which as a rule consist of a—in most cases solid—colloidally dispersed substance of long-chain molecular groups (e.g. gelatin, silica, polysaccharides) as the backbone-former and a liquid dispersant (e.g. water). The colloidally dispersed substance is often referred to as a thickener or gelling agent. It forms a three-dimensional network in the dispersant, it being possible for individual particles present in colloidal form to be linked to one another more or less firmly via electrostatic interaction. The dispersant which surrounds the network is distinguished by electrostatic affinity for the gelling agent, i.e. a predominantly polar (in particular: hydrophilic) gelling agent preferably gels a polar dispersant (in particular: water), whereas a predominantly nonpolar gelling agent preferably gels nonpolar dispersants.

Strong electrostatic interactions, which are realized, for example, in hydrogen bridge bonds between gelling agent and dispersant, but also between dispersant molecules amongst themselves, can lead to a high degree of crosslinking of the dispersant as well. Hydrogels can consist of virtually 100% of water (besides, for example, about 0.2-1.0% of a gelling agent) and have an entirely solid consistency. The water content is present here in ice-like structural elements, meaning that gels therefore do justice to the origin of their name [from Latin “gelatum”=“frozen” via the alchemistic term “gelatina” (16th Century) for the modern term “gelatin”].

In cosmetics and pharmaceutical technology, lipogels and oleogels (comprising waxes, fats and fatty oils), and also carbogels (comprising paraffin or petrolatum) are also common. In practice, a distinction is made between oleogels, which are virtually free from water, and hydrogels, which are virtually free from fat. In most cases, gels are transparent. In cosmetic or pharmaceutical technology, gels are usually characterized by a semisolid, often flowable, consistency.

In addition, so-called surfactant gels are customary preparations of the prior art. This is understood as meaning systems which, besides water, have a high concentration of emulsifiers, typically more than about 25% by weight, based on the total composition. If oil components are solubilized in these surfactant gels, microemulsion gels are obtained which are also referred to as “ringing gels”. By adding nonionic emulsifiers, for example alkyl polyglycosides, it is possible to obtain cosmetically more elegant microemulsion gels.

It is known per se to join together the droplets of a low viscosity, in particular thin-liquid, microemulsion using crosslinking substances in order, in so doing, to obtain the three-dimensional network of a gel.

Cream is understood as meaning a preparation which is not free-flowing and which has a viscosity, at 25° C., of more than 10 000 mPa*s (Viskotester VT-02, Haake). Lotion is understood as meaning a free-flowing preparation which has a viscosity, at 25° C., of 2000-10 000 mPa*s (Viskotester VT-02, Haake).

In addition, the preparations according to the invention can be used exceptionally well as impregnation medium for wipes and fabrics, which can be applied wet or dry by the consumer. The compositions with the ultrafine droplets readily attach to the fibers, which is to be regarded as an advantage.

It is also possible to provide the preparations according to the invention in the form of drug delivery systems. Drug delivery systems are plaster-like medicaments which contain the active ingredient in the form of a reservoir, from which the active ingredient release takes place over a prolonged period according to 0 order. On the basis of these properties, the drug delivery systems are able to supply the organism with constant amounts of active ingredient per unit of time over a long period irrespective of the active ingredient concentration in the reservoir and are therefore assigned to the group of delayed-release medicaments.

Drug delivery systems are distinguished between topical and transdermal delivery. The topical formulations comprise active ingredients whose release and effect is limited to the area directly below and the vicinity of the site of application. The transdermal formulations, by contrast, comprise active ingredients which are applied through the skin in order to produce, as a result of penetration into the vascular system, an active level of active ingredient in the entire organism. Compared with other medicament forms (e.g. ointments, sprays, suppositories, tablets), the drug delivery systems have a number of advantages:
Due to the long activity time of the drug delivery systems, patient compliance is considerably improved compared with medicaments which have to be applied more than once in the course of a day.
As a result of the application of the drug delivery systems, the dose can be reduced, thus giving rise to a reduction in the side effects of active ingredients which, for example, have a small therapeutic width.
Should side effects occur, application of the active ingredient can be stopped immediately by removing the drug delivery system.
As a result of the constant release of the active ingredient, the variations in active ingredient concentration which arise upon repeated application during the day are prevented in the area of skin and in the serum.
Compared with oral medicament forms, the transdermal application reduces the first-pass metabolism of the medicament since the metabolism in the area of skin is significantly lower compared with the metabolism in the stomach and liver. This results in the dose of the medicaments applied with drug delivery systems being lower compared with oral medicament forms.
In contrast to oral medicaments, with drug delivery systems it is not necessary to take into consideration how food influences penetration of the medicament.
The dosage can be defined very easily via the area of the drug delivery systems.

The preparations according to the invention are advantageously prepared by bringing a mixture of the basic components, comprising water phase, oil phase, one or more of the O/W emulsifiers according to the invention, if desired one or more coemulsifiers, and if desired further auxiliaries, additives or active ingredients, to a temperature which corresponds at least to the melting temperature of the highest-melting oil component, and then cooling the formed emulsion to room temperature, where, in accordance with the invention, the active ingredient or the active ingredients polidocanol and essential oils and any thickeners can be added at any time during the preparation. The entire operation is preferably carried out with stirring.

The examples below are intended to illustrate the present invention without limiting it. Unless stated otherwise, all amounts, proportions and percentages are based on the weight and the total amount or on the total weight of the preparations.

### EXAMPLES

O/W Emulsions

| | 1 | 2 | 3 | 4 | 5 |
|---|---|---|---|---|---|
| Cetyl alcohol | 1.0 | 0.5 | 1.0 | 2.0 | 3.0 |
| Glyceryl stearate | 1.0 | 1.5 | 1.0 | 3.0 | 2.0 |
| Paraffin oil | 2.5 | 1.0 | 4.0 | 2.5 | 1.5 |
| Decyl oleate | 0.5 | 0.75 | — | 2.0 | 0.25 |
| Octyldodecanol | — | 1.0 | — | 3.0 | 0.25 |
| Dicaprylyl carbonate | 0.1 | 0.05 | — | 0.5 | — |
| Glycerol | 3.0 | 5.0 | 12.0 | 15.0 | 1.5 |
| Dimethicone | 0.6 | 0.3 | 1.0 | 1.2 | 1.8 |
| Perfume | q.s. | q.s. | q.s. | q.s. | q.s. |
| Evening primrose oil | 2.0 | — | 1.0 | — | 8.0 |
| Caprylic/capric | 2.0 | 2.5 | 6.0 | 5.0 | 0.5 |
| triglyceride | | | | | |
| Methylparaben | 0.1 | 0.15 | — | 0.03 | 0.14 |
| Polidocanol | 1.0 | 1.0 | 2.0 | 2.5 | — |
| Menthol | 0.05 | — | 0.15 | 0.25 | 0.3 |
| Polyethylene glycol(12) | 1.0 | 2.2 | 2.6 | 2.4 | — |
| cetearyl ether | | | | | |
| Cyclomethicones | 0.5 | 0.75 | 1.0 | 2.0 | 0.25 |
| Ethanol | 1 | 5 | — | 3 | 2 |
| Modified starch | — | 2.5 | — | 0.15 | — |
| Propylparaben | 0.1 | 0.15 | — | 0.15 | — |
| Iodopropynyl | — | — | 0.3 | — | 0.1 |
| butylcarbamate + | | | | | |
| DMDM hydantoin | | | | | |
| Water | ad 100 | ad 100 | ad 100 | ad 100 | ad 100 |

O/W Emulsions

| | 6 | 7 | 8 | 9 | 10 |
|---|---|---|---|---|---|
| PEG-40 stearate | — | 0.5 | — | — | 1.0 |
| Panthenol | — | 0.75 | 1.0 | 2.0 | 0.25 |
| Octyldodecanol | — | 1.0 | 0.75 | 3.0 | 0.25 |
| Dicaprylyl carbonate | — | 0.05 | 0.15 | 0.5 | 1.0 |
| Glycerol | 3.0 | 5.0 | 10.0 | 15.0 | 1.5 |
| Lactic acid | 0.6 | 0.3 | 0.15 | 1.2 | 1.8 |
| Perfume | q.s. | q.s. | q.s. | q.s. | q.s. |
| Polidocanol | 3.0 | 1.0 | 0.5 | 2.5 | 5.0 |
| Menthol | 0.1 | 1.0 | 0.5 | 0.25 | 0.5 |
| Cetyl alcohol | 1.0 | 0.5 | 1.0 | 2.0 | 3.0 |
| Glyceryl stearate | 1.0 | 1.5 | 1.0 | 3.0 | 2.0 |
| Potassium sorbate | — | — | 0.3 | — | 0.15 |
| Caprylic/capric | — | 2.5 | 3.0 | 5.0 | 0.5 |
| triglyceride | | | | | |
| Methylparaben | — | 0.15 | 0.05 | 0.3 | 0.4 |
| Propylparaben | — | 0.4 | 0.25 | 0.15 | — |
| Water | ad 100 | ad 100 | ad 100 | ad 100 | ad 100 |

O/W Emulsion

| | 11 | 12 | 13 | 14 | 15 |
|---|---|---|---|---|---|
| Cetyl alcohol | 1.0 | 0.5 | 1.0 | 2.0 | 3.0 |
| Glyceryl stearate | 1.0 | 1.5 | 1.0 | 3.0 | 2.0 |
| Polidocanol | 1.0 | 1.0 | 1.5 | 0.5 | 0.75 |
| Menthol | 0.5 | 0.1 | 0.05 | 0.25 | 0.2 |
| Paraffin oil | 2.5 | — | 8.0 | 2.5 | 1.5 |
| Cocoglyceride | 8.0 | 6.0 | 0.5 | — | 2.5 |
| PEG-40 hydrogenated | 1.0 | 1.5 | — | 1.8 | — |
| castor oil | | | | | |
| Hydrogenated | — | 0.75 | 1.0 | 2.0 | 0.25 |
| polyisobutene | | | | | |
| Octyldodecanol | 0.5 | 1.0 | 0.75 | 8.0 | 0.25 |
| Dicaprylyl carbonate | 0.1 | 0.05 | 0.15 | 0.5 | 1.0 |
| Glycerol | 3.0 | 5.0 | — | 15.0 | 10.0 |
| Perfume | q.s. | q.s. | q.s. | q.s. | q.s. |
| Butylene glycol | 5.0 | — | 15.0 | — | 8.0 |
| Caprylic/capric | 2.0 | 2.5 | 3.0 | 5.0 | 0.5 |
| triglyceride | | | | | |
| Methylparaben | 0.4 | — | 0.05 | 0.3 | 0.4 |
| Propylparaben | 0.3 | 0.4 | 0.25 | 0.15 | — |
| Water | ad 100 | ad 100 | ad 100 | ad 100 | ad 100 |

O/W Emulsions

| | 16 | 17 | 18 | 19 | 20 |
|---|---|---|---|---|---|
| Polyethylene glycol(12) | 1.0 | 0.5 | — | — | — |
| cetearyl ether | | | | | |
| Cyclomethicone | 0.5 | 0.75 | 1.0 | 2.0 | 0.25 |
| Octyldodecanol | 0.5 | 1.0 | 0.75 | 3.0 | 0.25 |
| Dicaprylyl carbonate | 0.1 | 1.0 | 15.0 | 0.5 | 1.0 |
| Dimethicone | — | — | — | 5.0 | 1.0 |
| Glycerol | 3.0 | 5.0 | 10.0 | 15.0 | 1.5 |
| Polidocanol | 1.0 | 1.0 | 5 | 2.5 | 10 |
| Lanolin alcohol | 0.25 | 0.5 | 1.5 | 3.0 | 1.0 |
| Menthol | 0.05 | 0.5 | 0.25 | 0.3 | 0.5 |
| Paraffin oil | 2.5 | 1.0 | 8.0 | 2.5 | 1.5 |
| Perfume | q.s. | q.s. | q.s. | q.s. | q.s. |
| Tocopherol | 1.0 | 0.5 | 1.5 | 3.0 | 0.5 |
| Caprylic/capric | 2.0 | 2.5 | 3.0 | 5.0 | 0.5 |
| triglyceride | | | | | |
| Methylparaben | 0.4 | 0.15 | 0.05 | 0.3 | 0.4 |
| Propylparaben | 0.3 | 0.4 | 0.25 | 0.15 | — |
| Modified starch | — | 2.5 | — | 0.15 | 5.0 |
| Water | ad 100 | ad 100 | ad 100 | ad 100 | ad 100 |

Following application to the irritated skin, all of the example preparations listed exhibit an immediate reduction in itchiness ranging to a loss of itchiness. In addition, the preparations according to the invention can be applied easily to the skin and can be spread in a pleasant manner.

1. A cosmetic or dermatological preparation comprising
water,
at least one oil-in-water (O/W) emulsifier,
at least one oil phase,
polidocanol, and
at least one essential oil.

2. The preparation as claimed in claim 1, further comprising at least one coemulsifier.

3. The preparation as claimed in claim 2, wherein the at least one coemulsifier includes glyceryl stearate.

4. The preparation as claimed in claim 1, wherein the at least one O/W emulsifier includes monostearate polyoxyethylene (40).

5. The preparation as claimed in claim 1, wherein the at least one O/W emulsifier is selected from the group consisting of ethoxylated fatty alcohols, ethoxylated stearates and glycerol esters.

6. The preparation as claimed in claim 1, wherein the at least one essential oil includes menthol.

7. The preparation as claimed in claim 1, wherein the total amount of polidocanol is from 0.01 to 20% by weight, based on the total weight of the preparation.

8. The preparation as claimed in claim 1, wherein the total amount of the oil phase is from 1 to 50% by weight, based on the total weight of the preparation.

9. The preparation as claimed in claim 1, further comprising ribwort tincture.

10. The preparation as claimed in claim 1, further comprising at least one moisturizer.

11. The preparation as claimed in claim 1, further comprising at least one cosmetic or dermatological auxiliary selected from the group consisting of dyes, pigments, powder substances, hydrophilic or lipophilic active ingredients, deodorants, and photoprotective agents.

12. The preparation as claimed in claim 11, wherein the at least one cosmetic or dermatological auxiliary is selected from the group consisting of antioxidants, NO synthase inhibitors, catechins, gallic esters, flavones, flavonoids, ubiquinones, plastoquinones, creatines, creatine derivatives, and repellents.

13. The preparation as claimed in claim 1, further comprising coenzyme Q10.

14. The preparation as claimed in claim 1, further comprising at least one substance selected from the group consisting of photoprotective filter substance capable of absorbing UV-A, UV-B, or UV-A and UV-B radiation.

15. The preparation as claimed in claim 1, wherein the total amount of polidocanol is from 1 to 10% by weight, based on the total weight of the preparation.

16. The preparation as claimed in claim 1, wherein the total amount of the at least one essential oil is from 0.001 to 10% by weight, based on the total weight of the preparation.

17. The preparation as claimed in claim 1, wherein the total amount of the at least one essential oil is from 0.01 to 1% by weight, based on the total weight of the preparation.

18. The preparation as claimed in claim 1, wherein the total amount of the oil phase is from 1 to 40% by weight, based on the total weight of the preparation.

19. The preparation as claimed in claim 10, wherein the at least one moisturizer is selected from the group consisting of polyols.

20. The preparation as claimed in claim 10, wherein the at least one moisturizer is selected from the group consisting of glycerol, sorbitol, urea, and butylene glycol.

21. The preparation as claimed in claim 1, further comprising at least one acylcarnitine.

22. The preparation as claimed in claim 21, wherein the at least one acylcarnitine includes acetylcarnitine.

23. The preparation as claimed in claim 1, further comprising L-carnitine.

24. A cosmetic or dermatological preparation comprising at least one nonionic emulsifier, polidocanol, and menthol.

25. The preparation as claimed in claim 24, wherein the at least one nonionic emulsifier includes polyethylene glycol (12) cetearyl ether.

26. The preparation as claimed in claim 1, wherein the preparation is in the form of a cream, lotion, gel, spray, or foam.

27. A drug delivery system comprising a preparation comprising at least one O/W emulsifier, water, at least one oil phase, polidocanol, and at least one essential oil.

28. The drug delivery system as claimed in claim 27, wherein the preparation further comprises at least one coemulsifier.

29. The drug delivery system as claimed in claim 28, wherein the at least one coemulsifier includes glyceryl stearate.

30. The drug delivery system as claimed in claim 27, wherein the at least one O/W emulsifier includes monostearate polyoxyethylene (40).

31. The drug delivery system as claimed in claim 27, wherein the at least one O/W emulsifier is selected from the group consisting of ethoxylated fatty alcohols, ethoxylated stearates and glycerol esters.

32. The drug delivery system as claimed in claim 27, wherein the at least one essential oil includes menthol.

33. The drug delivery system as claimed in claim 32, wherein the at least one O/W emulsifier includes polyethylene glycol (12) cetearyl ether.

34. The drug delivery system as claimed in claim 27, further comprising ribwort tincture.

35. The drug delivery system as claimed in claim 27, further comprising coenzyme Q10.

36. The drug delivery system as claimed in claim 27, further comprising at least one acylcarnitine.

37. The drug delivery system as claimed in claim 36, wherein the at least one acylcarnitine includes acetylcarnitine.

38. The drug delivery system as claimed in claim 27, further comprising L-carnitine.

39. A dermatological wipe comprising a fabric or wipe impregnated with a dermatological preparation comprising water, at least one O/W emulsifier, at least one oil phase, polidocanol, and at least one essential oil.

40. A method for treating the skin or scalp comprising applying a dermatological preparation comprising water, at least one O/W emulsifier, at least one oil phase, polidocanol, and at least one essential oil.

41. The method as claimed in claim 40, wherein the dermatological preparation further comprises at least one coemulsifier.

42. The method as claimed in claim 41, wherein the at least one coemulsifier includes glyceryl stearate.

43. The method as claimed in claim 40, wherein the at least one O/W emulsifier includes monostearate polyoxyethylene (40).

44. The method as claimed in claim 40, wherein the at least one O/W emulsifier is selected from the group consisting of ethoxylated fatty alcohols, ethoxylated stearates and glycerol esters.

45. The method as claimed in claim 40, wherein the at least one essential oil includes menthol.

46. The method as claimed in claim 40, wherein the dermatological preparation further comprises ribwort tincture.

47. The method as claimed in claim 40, wherein the dermatological preparation further comprises at least one cosmetic or dermatological auxiliary selected from the group consisting of antioxidants, NO synthase inhibitors, catechins, gallic esters, flavones, flavonoids, ubiquinones, plastoquinones, creatines, creatine derivatives, and repellents.

48. The method as claimed in claim 40, wherein the dermatological preparation further comprises coenzyme Q10.

49. The method as claimed in claim 40, wherein the dermatological preparation further comprises at least one polyol.

50. The method as claimed in claim 40, wherein the dermatological preparation further comprises at least one acylcarnitine.

51. The method as claimed in claim 40, wherein the dermatological preparation further comprises L-carnitine.

52. The method as claimed in claim 40, wherein the at least one O/W emulsifier includes polyethylene glycol (12) cetearyl ether.

53. A method for reducing or relieving itchiness on the skin or scalp comprising applying to the itchy area of the skin or scalp a dermatological preparation comprising water, at least one O/W emulsifier, at least one oil phase, polidocanol, and at least one essential oil.
